(19) United States
(12) Patent Application Publication
(10) Pub. No.: US 2005/0214740 A1
(43) Pub. Date: Sep. 29, 2005

(54) Device for measuring extracellular potential and manufacturing method of the same

(75) Inventors: Hiroshi Ushio, Moriguchi-shi (JP); Masaya Nakatani, Takarazuka-shi (JP); Hiroaki Oka, Hirakata-shi (JP); Nobuhiko Ozaki, Ikoma-shi (JP)

Correspondence Address: STEPTOE & JOHNSON LLP, 1330 CONNECTICUT AVENUE, N.W., WASHINGTON, DC, 20036, US

(73) Assignee: Matsushita Elec. Ind. Co., Ltd., Kadoma-shi (JP)

(21) Appl. No.: 11/081,759

(22) Filed: Mar. 17, 2005

(30) Foreign Application Priority Data
Mar. 22, 2004 (JP) 2004-82240

(51) Int. Cl. C12Q 1/00; C12P 21/06

(52) U.S. Cl. 435/4

(57) ABSTRACT

A device for measuring extracellular potential includes a diaphragm and a detecting electrode. The diaphragm is formed on one surface thereof with a depression including at least one curved surface, and a hole is provided so as to penetrate from the curved surface of the depression through to the other surface of the diaphragm. An opening on the curved surface of the depression is smaller than an opening on the other side, and a detecting electrode is provided on the wall surface of the hole.

### BACKGROUND OF THE INVENTION

1. Field of the Invention

The present invention relates to a device for measuring extracellular potential used for measuring the extracellular potential or for measuring physico-chemical change generated by cellular activities and a method of manufacturing the same.

2. Description of the Related Art

In the related art, a candidate substance for a medicine is screened by a patch clamp technique or a method using a fluorescent pigment or a luminous indicator with electrical activities of cells as a reference mark. In the patch clamp technique, transportation of ion via a single channel protein molecule is electrically recorded by a fine electrode probe using a small portion (a patch) of cell membrane attached to the distal portion of a micropipette. This technique is one of a few-number of methods which can investigate the function of a single protein molecule on real time basis. There are also methods of measuring an electric activity of a cell by monitoring the movement of ion in the cell by the fluorescent pigment or the luminous indicator emitting light according to the change of the concentration of a specific ion.

However, since the patch clamp technique requires a special technique for preparation and operation of the micropipette, much time is required for measuring one sample. Therefore, this technique is not suitable for an application which requires high-speed screening which treats a large amount of candidate compounds for a medicine. On the other hand, the method using the fluorescent pigment can screen a large amount of candidate compounds for a medicine at a high speed. However, a step of dying the cell is required, and a back ground level detected at the time of measurement becomes higher due to the influence of the used pigment, and the pigment is faded out with time. Therefore, the S/N ratio is disadvantageously deteriorated.

An alternative method is disclosed in International Publication WO 02/055653. According to this method, a device for measuring the extracellular potential is employed. The device uses a substrate which includes a cell holding portion, and an electrode provided on this substrate. In this method, high quality data can be obtained as well as data obtained by the patch clamp technique and, in addition, a large amount of samples can be measured easily at a high speed as with the method using the fluorescent pigment. The device includes at least one well having a cell holding portion provided on the substrate, and a sensor for detecting electric signals provided in this well. Referring to the drawings, the operation of the device for measuring extracellular potential will be described in detail hereinafter.

FIG. 17 is a type cross-sectional view showing a structure of the well of the device for measuring extracellular potential in the related art. Culture solution 20 is filled in well 14, and test cell (hereinafter, referred to as “cell”) 19 is captured or held by a cell holding portion provided on substrate 16. The cell holding portion includes depression 15 formed on substrate 16 and through-hole 17 in communication with depression 15 via an opening. Disposed in through-hole 17 is measuring electrode (hereinafter, referred to as “electrode”) 18, which is a sensor portion, and electrode 18 is connected to a signal detector via wiring. In the case of measurement, cell 19 is tightly held in depression 15 by sucking cell 19 from the side of through-hole 17 with a suction pump or the like. In this manner, an electric signal generated by the activity of cell 19 is detected by electrode 18 provided on the side of through-hole 17 without leaking into culture solution 20 in well 14.

However, in the structure of the related art as described above, since electrode 18 is formed inside through-hole 17, measurement may be unstable. When well 14 is downsized, the amount of filled culture solution 20 interposed between cell 19 and electrode 18 becomes insufficient. From these problems, it is difficult to achieve measurement with high degree of accuracy.

### SUMMARY OF THE INVENTION

A device for measuring extracellular potential of the present invention includes a diaphragm and a detecting electrode. The diaphragm is formed on one surface thereof with a depression including at least one curved surface, and a hole is provided so as to penetrate from the curved surface of the depression through to the other surface of the diaphragm. An opening on the curved surface of the depression is smaller than the opening on the other side, and the detecting electrode is provided on the wall surface of the hole. Since the device for measuring extracellular potential has the detecting electrode on the wall surface of the hole, the accuracy of measurement of the extracellular potential is high, and the physico-chemical change which occurs during activity of the cell can be detected efficiently and stably with high degree of accuracy.

### BRIEF DESCRIPTION OF THE DRAWINGS

FIG. 1 is a perspective view of a device for measuring extracellular potential according to an exemplary embodiment of the present invention.

FIG. 2 is a cross-sectional view of the device shown in FIG. 1 taken along the line 2-2.

FIG. 3 is an enlarged plan view of a peripheral portion of a through-hole of the device shown in FIG. 1.

FIG. 4 is an enlarged cross-sectional view showing a essential portion for explaining the action of the device shown in FIG. 1.

FIGS. 5 to 10 are cross-sectional views for explaining a method of manufacturing the device shown in FIG. 1.

FIG. 11 is a cross-sectional view of another substrate for manufacturing the device for measuring extracellular potential according to the exemplary embodiment of the invention.

FIG. 12 is a cross-sectional view of another device for measuring extracellular potential according to the exemplary embodiment of the invention.

FIG. 13 is an enlarged plan view of the peripheral portion of the through-hole of the device shown in FIG. 12.

FIG. 14 is a cross-sectional view of a still other device for measuring extracellular potential according to the exemplary embodiment of the invention.

FIG. 15 is an enlarged plan view of the peripheral portion of the through-hole of the device shown in FIG. 14.

FIG. 16 is a cross-sectional view for explaining a method of manufacturing the device shown in FIG. 14.

FIG. 17 is a cross-sectional view of the device for measuring extracellular potential in the related art.

### DETAILED DESCRIPTION OF EXEMPLARY EMBODIMENT

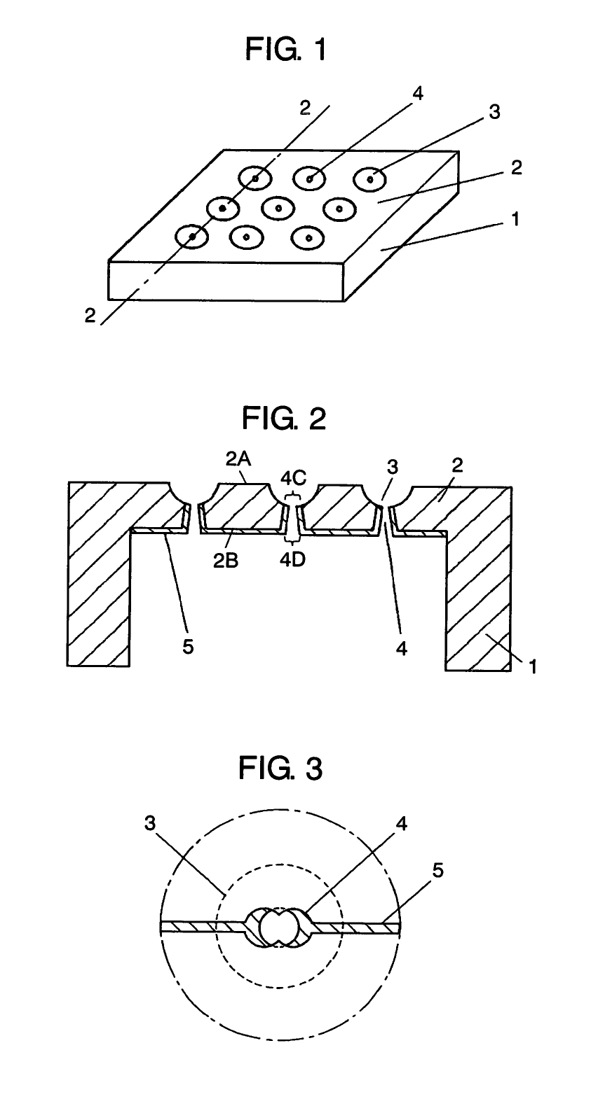

FIG. 1 is a perspective view of a device for measuring extracellular potential according to an exemplary embodiment of the present invention, FIG. 2 is a cross-sectional view of the device shown in FIG. 1 taken along the line 2-2, and FIG. 3 is an enlarged plan view of a peripheral portion of a through-hole of the device shown in FIG. 2 when viewed from the side of the lower surface of the substrate.

The structure of the device for measuring extracellular potential according to the invention will be described. Substrate 1 is formed of silicon, and diaphragm 2 is disposed at the upper surface (first surface) side of substrate 1. The material of diaphragm 2 is silicon same as substrate 1. Depression 3 is provided on upper surface 2A side (first surface side) of diaphragm 2, and is formed of a semispherical curved surface. Through-hole (hereinafter, referred to as “hole”) 4 continues from a predetermined position on the curved surface of depression 3 to lower surface 2B side (second surface side) opposing to upper surface 2A of diaphragm 2.

Opening 4C of hole 4 provided on the curved surface of depression 3 at the predetermined position is smaller than opening 4D provided on lower surface 2B side of diaphragm 2, and the wall surface of hole 4 has a linear shape. In other words, a ridge line of the wall surface connecting opening 4C formed on the curved surface of depression 3 at the predetermined position and opening 4D provided on lower surface 2B side of diaphragm 2 is a straight line. In this manner where the silicon is used for substrate 1, the device for measuring extracellular potential formed with diaphragm 2, depression 3, and hole 4 by dry etching with high degree of accuracy is obtained.

In addition, detecting electrode (hereinafter, referred to as “electrode”) 5 is provided on the wall surface of hole 4 and lower surface 2B of diaphragm 2. Electrode 5 is formed mainly of noble metal in terms of safety of measurement. With the provision of electrode 5 on the wall surface of hole 4, the accuracy of measurement of the extracellular potential can be improved. In addition, by employing noble metal, electrode 5 having less possibility of formation of the oxide film and showing a stable electrode characteristic can be obtained. The noble metal preferably used for electrode 5 is gold or platinum.

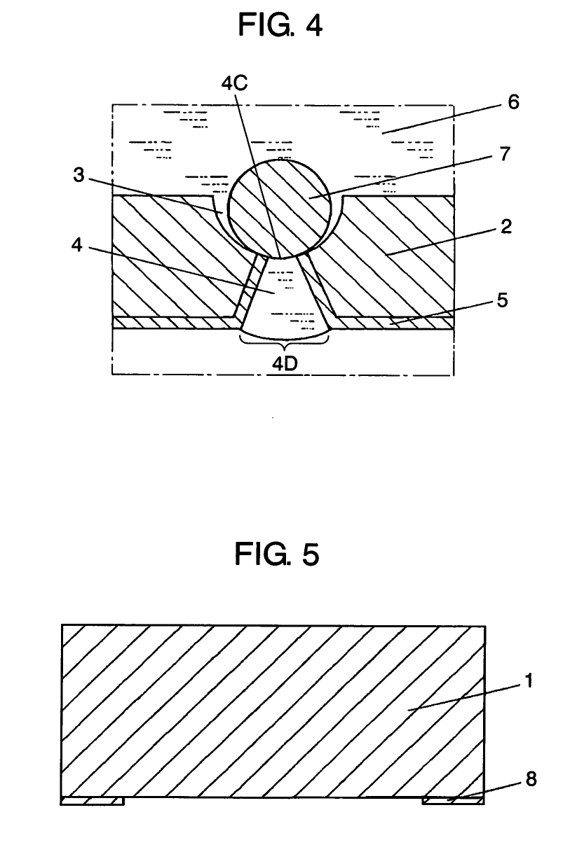

Referring now to FIG. 4, the operation of the device for measuring extracellular potential according to the exemplary embodiment of the invention will be described herein after. FIG. 4 is an enlarged cross-sectional view of the device for measuring extracellular potential showing a position on diaphragm 2 formed with depression 3, hole 4, and electrode 5. At first, a procedure for detecting the physico-chemical change of culture solution will be described.

When the upper portion of diaphragm 2 is filled with culture solution 6, depression 3 and hole 4 are filled with culture solution 6 in sequence by surface tension of culture solution 6. When the upper space of diaphragm 2 is pressurized, or the lower space of diaphragm 2 is depressurized, culture solution 6 is injected from hole 4 to opening 4D on the side of lower surface 2B of diaphragm 2. At this time, when the condition of the pressurization or depressurization is adjusted to an adequate value, culture solution 6 is formed into a meniscus shape at the distal end of opening 4D and is brought into a static state as shown in FIG. 4.

Next, a procedure for measuring the extracellular potential of test cell (hereinafter, referred to as “cell”) 7 or the physico-chemical change of cell 7 will be described. As shown in FIG. 4, when cell 7 is put together with culture solution 6 and the upper space of diaphragm 2 is pressurized, or the lower space of diaphragm 2 is depressurized, cell 7 and culture solution 6 are pulled into depression 3 together. Depression 3 is preferably formed of a predetermined curved surface into a shape further efficient for holding cell 7.

Then, the upper and lower pressure is adjusted so that culture solution 6 forms an adequate meniscus at opening 4D, which is the exit side of hole 4, after cell 7 is held in depression 3. Cell 7 is held in depression 3 so as to block up opening 4C on the depression side of hole 4. Subsequently, a behavior for giving stimulation to cell 7 is performed. The type of simulation may be, for example, mechanical displacement, or physical stimulation such as light, heat, electricity, or electromagnetic wave, in addition to chemical stimulation using a chemical medicament or poison.

When cell 7 reacts actively against such stimulation, for example, cell 7 discharges or absorbs various types of ion thorough an ion channel which the cell membrane possesses. Although the reaction occurs at positions where cell 7 is in contact with culture solution 6 directly, but ion exchange also occurs indirectly between culture solution 6 in hole 4 and cell 7.

As a consequence, the ion concentration of culture solution 6 in hole 4 and the ion concentration of intracellular fluid of cell 7 change, and hence electrode 5 can detect the change via culture solution 6. In this case, since electrode 5 is also formed on the entire wall surface of hole 4, it is formed to the extent near the position where cell 7 is held. Therefore, electrode 5 can perform measurement of the change of the extracellular potential of cell 7 or the physico-chemical change which is generated by cell 7 with high degree of accuracy without being influenced by noise, even by performing measurement of the extracellular potential via culture solution 6.

Referring now to FIG. 5 to FIG. 11, a method of manufacturing the device for measuring extracellular potential with the structure shown in FIG. 2 will be described. FIGS. 5 to 11 are cross-sectional views for explaining the process in a method of manufacturing the device for measuring extracellular potential shown in FIG. 2.

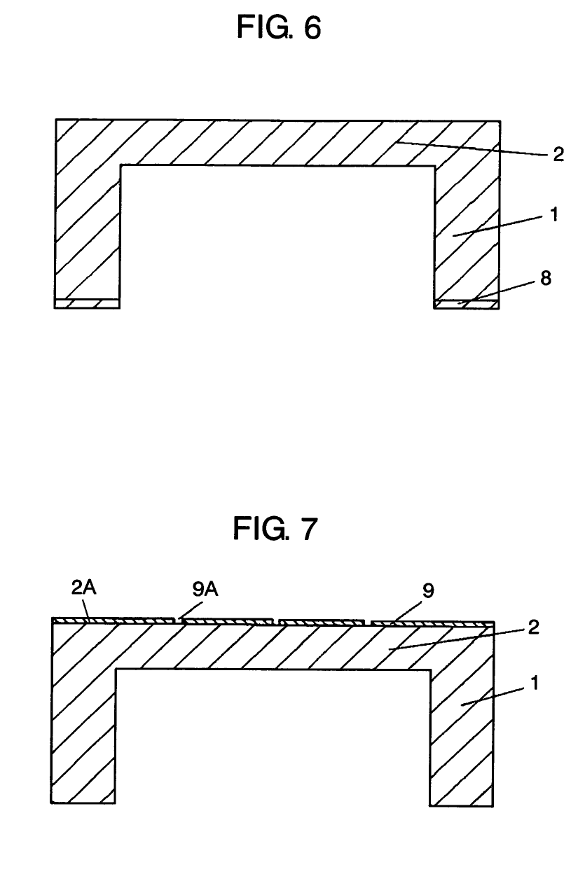

As shown in FIG. 5, in the method of manufacturing the device for measuring extracellular potential, substrate 1 formed of silicon is prepared, and resist mask 8 is formed on the lower surface of substrate 1. Subsequently, diaphragm 2 is formed on top of substrate 1 by etching from the lower surface side of substrate 1 as shown in FIG. 6. In order to form diaphragm 2 into a predetermined thickness, the amount of etching should be controlled with high degree of accuracy. In particular, when substrate 1 is silicon, excessive etching may result in penetration of diaphragm 2.

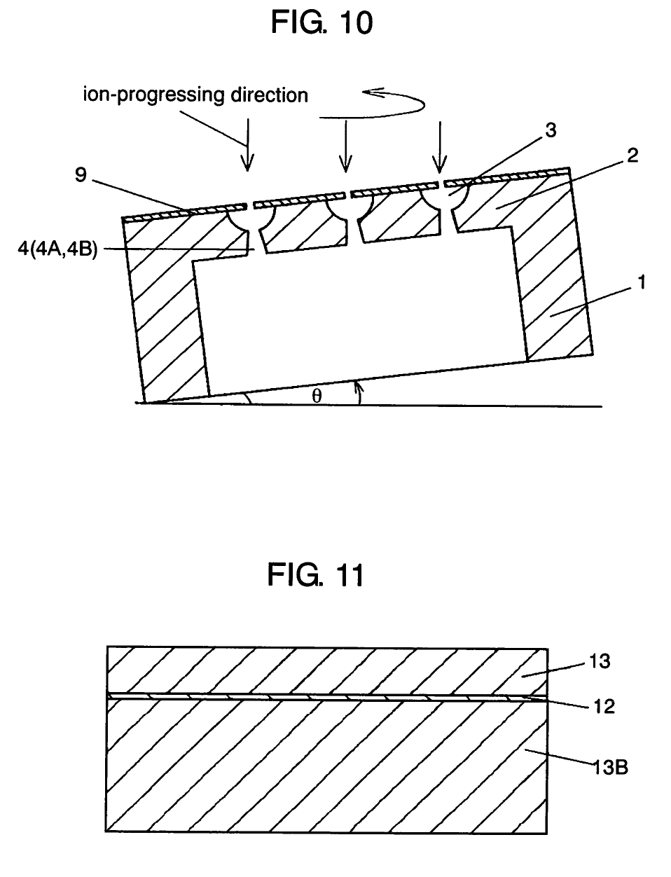

In order to solve such a problem, it is also possible to use material having a layered structure including silicon layers 13, 13B, and silicon dioxide layer 12 interposed between the silicon layers, as shown in FIG. 11 as a material for substrate 1. Such a substrate is referred to as SOI (Silicon On Insulator) substrate, a thickness of silicon layer 13 is previously determined, and the etching speed is significantly lowered at silicon dioxide layer 12. Therefore, the separate working for the thickness of diaphragm 2 is not needed, and thereby the thickness of diaphragm 2 can be formed with high degree of accuracy. When such a material is used, it is necessary to remove exposed silicon dioxide layer 12 in a known method after having formed diaphragm 2, if needed. As a result, the device for measuring extracellular potential with high degree of accuracy and superior in productivity, and the method of manufacturing the same are achieved by using the SOI substrate.

Subsequently, resist mask 8 is removed after having formed diaphragm 2. Then, as shown in FIG. 7, resist mask 9 is formed on upper surface 2A side of diaphragm 2. The shape of etching hole 9A of resist mask 9 at this time is designed so as to be substantially the same as the shape of through-hole (hereinafter, referred to as “hole”) 4A, which is required and will be described later.

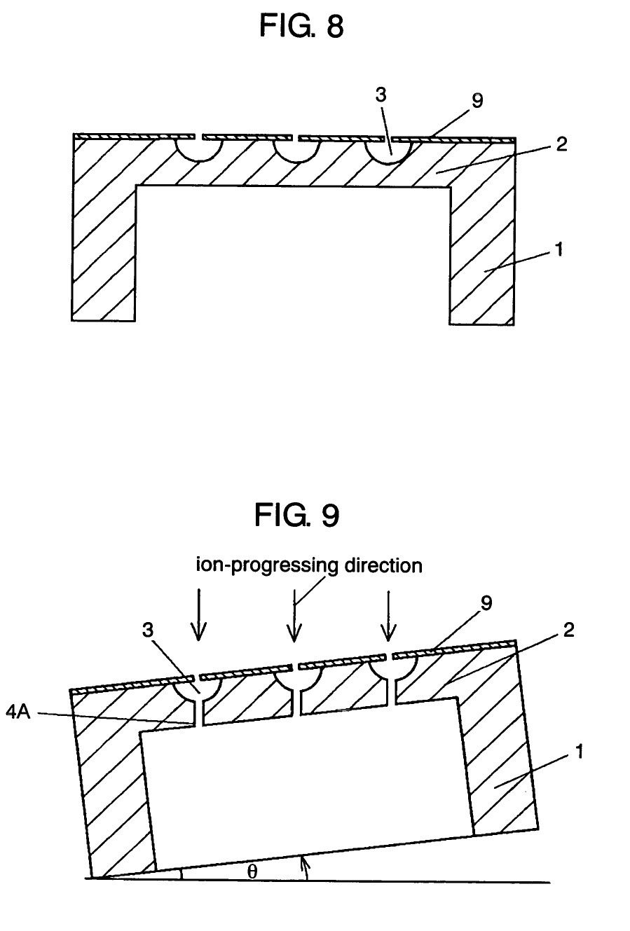

Subsequently, as shown in FIG. 8, dry etching is performed from the side of diaphragm 2. At this time, only gas which promotes etching is used as etching gas. When substrate 1 is formed of silicon, gas such as SF₆, CF₄ and XeF₂ can be used as the gas for promoting etching. These gases act to promote the etching of silicon as substrate 1 in the lateral direction as well as the depth direction. The effect is checked out by using XeF₂ experimentally. Accordingly, the shape of etching is semispherical shape about the opening of etching hole 9A as shown in FIG. 8, and depression 3 is formed. Since resist mask 9 is etched little, it keeps the initial shape.

Subsequently, as shown in FIG. 9, hole 4A is formed to diaphragm 2 of substrate 1 by an ionic dry etching. In this case, substrate 1 is installed so as to incline by an angle θ, which is larger than 0° with respect to a plane perpendicular to the ion-progressing direction, and forms hole 4A. When forming hole 4A, dry etching is performed by repeating the etching process using gas which promotes etching and the process of forming a protective film on the etched inner wall alternately. In this case, gas for promoting the etching and gas for restraining etching are alternately used as etching gas in each process. The gas for promoting etching includes SF₆, CF₄, and XeF₂. The gas for restraining etching includes CHF₃, C₄F₈. After having performed etching to a small extent using such gas for promoting etching, a protective film of polymer of CF₂ is formed on the etched wall surface by gas for restraining etching. By the dry etching method which repeats these processes alternately, hole 4A can be formed progressively linearly only downwardly of etching hole 9A provided on resist mask 9.

The mechanism of dry etching which progresses only downwardly of etching hole 9A will be described further in detail. In the etching process using gas for promoting etching, a high-frequency wave is applied on substrate 1 which is capacity-coupled with a high-frequency power source in a plasma generating area formed by the inductively coupling method using an external coil. Accordingly, a negative bias voltage is generated on substrate 1, and SF₅⁺ or CF₃⁺ which are positive ions in plasma collide against substrate 1. Consequently, etching operation progresses vertically and downwardly. In the process of forming the protective film by gas for restraining etching, plasma is generated by the inductively coupling method in the same manner. However, since a high-frequency wave is not applied to substrate 1 in the range of plasma generating area, a bias voltage is not generated at all on substrate 1. Therefore, CF⁺, which exists in the plasma and is a material of the protective film, is not subjected to deflection, and hence a uniform protective film is formed on the etched wall surface of substrate 1. In an experiment, verification is made by using SF₆ as gas for promoting etching, and C₄F₈ as gas for restraining etching. By repeating such a process alternately, etching progresses only vertically downwardly, and hence linear hole 4A as shown in FIG. 9 is formed as a result.

For example, when the depth of depression 3 is 10 μm, the depth of hole 4A is 10 μm, the diameter of etching hole 9A is 5 μm, the condition in which substrate 1 is installed in a state of being inclined by an angle larger than 0° and at most 7° with respect to the plane perpendicular to the ion-progressing direction is most efficient.

Subsequently, as shown in FIG. 10, etching is performed after rotating substrate 1 by 180° within upper surface 2A of diaphragm 2, so that through-hole (hereinafter, referred to as “hole”) 4B is formed. Here, by setting an angle θ of inclination with respect to the plane perpendicular to the ion-progressing direction to a predetermined angle, holes 4A and 4B join together as shown in FIG. 10. Consequently, the combination of holes 4A and 4B is formed into a structure of hole 4 shown in FIG. 2. Therefore, opening 4C formed at a predetermined position on the curved surface of depression 3 is formed into a smaller shape than opening 4D provided on lower surface 2B side of diaphragm 2. The ridge line of the wall surface of hole 4 has a linear structure.

Subsequently, electrode 5 formed of gold layer and titan layer is formed by a normal thin-film forming method on the entire wall surface of hole 4 and lower surface 2B of diaphragm 2 to form a structure as shown in FIG. 2. At this time, opening 4D of hole 4 is larger than opening 4C, and the ridge line of the wall surface of hole 4 extends linearly. Therefore, by forming electrode 5 using a thin-film forming method from the lower surface side of substrate 1, electrode 5 can easily be formed continuously to opening 4C without discontinuation.

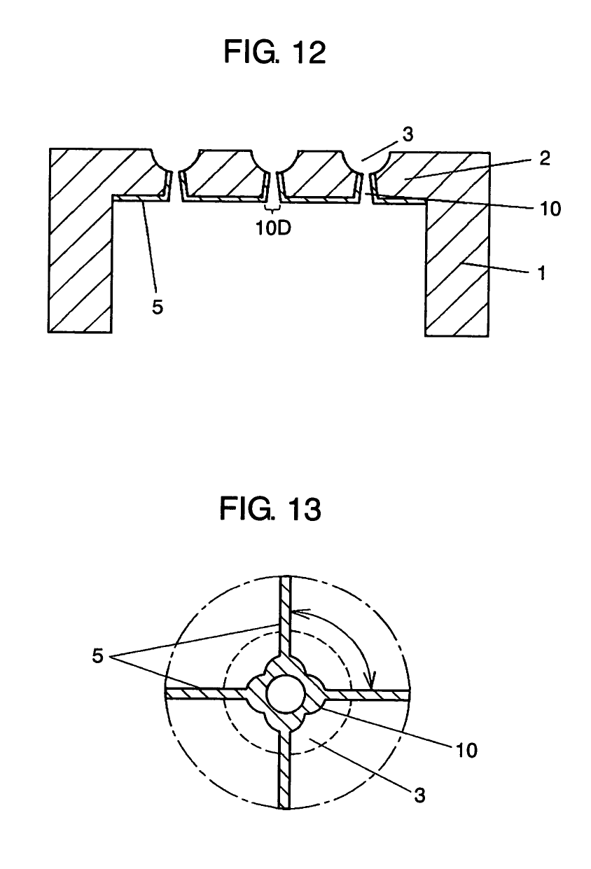

In the process of forming the through-hole, as shown in FIG. 9, it is also possible to rotate substrate 1 within upper surface 2A of diaphragm 2 at an angle other than 180° after etching is performed. For example, as shown in FIG. 12 and FIG. 13, through-hole 10 can be formed also by rotating by 90° each and subsequently performing etching. In this method, the shape of opening 10D formed on the lower side of diaphragm 2 can be freely designed, and the area of opening 10D can also be increased. Accordingly, cell 7 can easily be held since a pressure is efficiently transferred upon sucking from a lower direction. By employing 360°/n (n designates positive integer) as an angle of rotational movement, the symmetric property of the cross-sectional shape of through-hole 10 is improved, thereby further facilitating to hold cell 7 and increasing productivity.

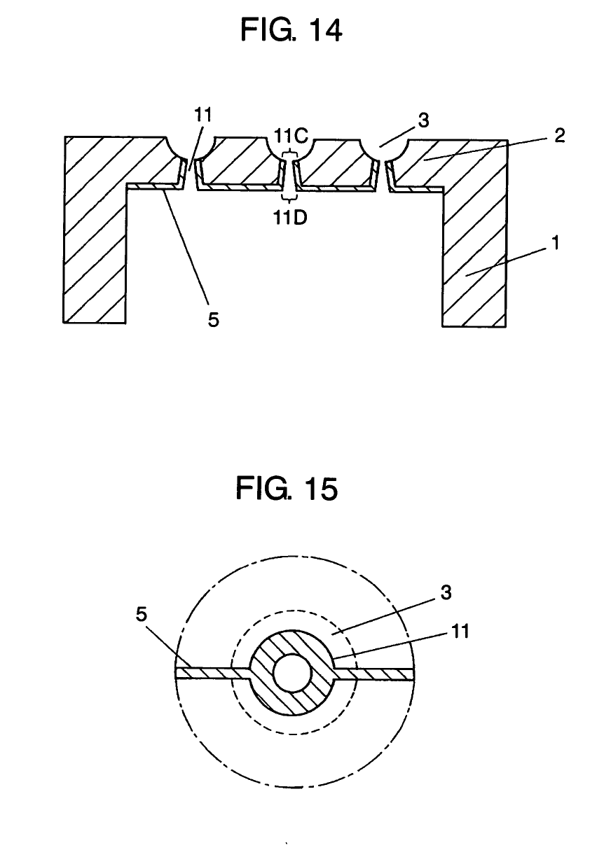

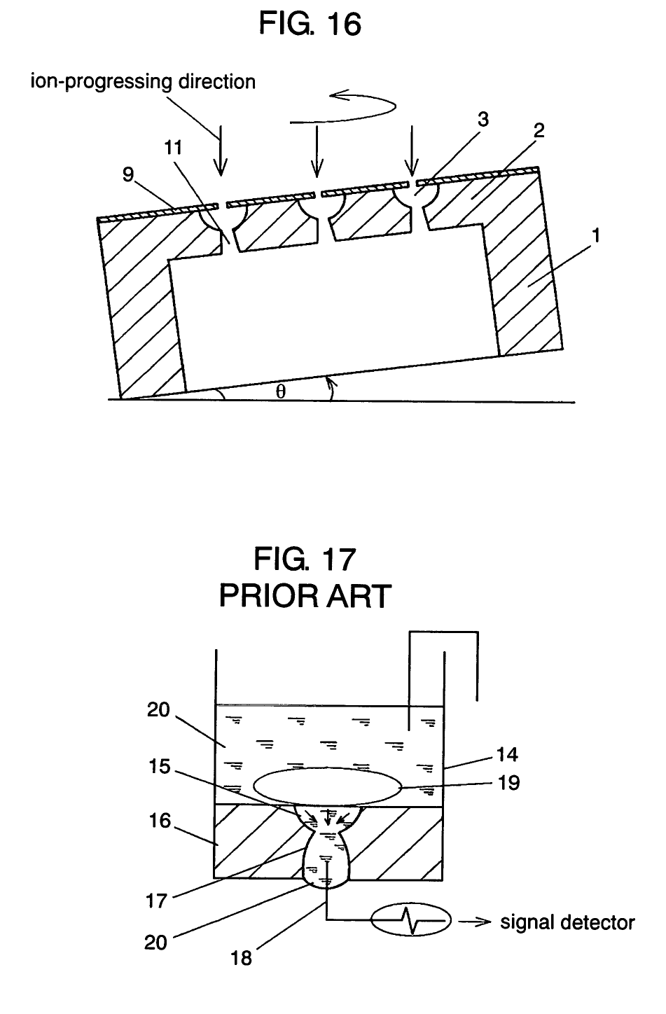

Next, another method of forming the through-hole will be described. FIG. 14 is a cross-sectional view of a still other device for measuring extracellular potential according to the exemplary embodiment of the invention, FIG. 15 is an enlarged view of the peripheral portion of through-hole (hereinafter, referred to as “hole”) 11, and FIG. 16 is a cross-sectional view for explaining the manufacturing process.

In the device shown in FIGS. 14 and 15, the shape of hole 11 is a circular truncated conical shape. A method of manufacturing the same will be described. Since depression 3 can be formed in the same process as the process of manufacturing shown in FIG. 5 to FIG. 8 in the exemplary embodiment, detailed description is omitted.

Subsequently, as shown in FIG. 16, hole 11 of the circular truncated conical shape is formed. At this time, substrate 1 is inclined by an angle θ which is larger than 0° with respect to the plane perpendicular to the ion-progressing direction, and dry etching is performed while rotating the same continuously in upper surface 2A of diaphragm 2. In such a dry etching process, opening 11C provided at a predetermined position on the curved surface of depression 3 and opening 11D provided on lower surface 2B side are formed into a circular shape. Then, circular hole 11 having a smaller diameter at opening 11C than that at opening 11D is formed. The ridge line of the wall surface of hole 11 extends linearly.

Preferably, diaphragm 2 is rotated at least by one revolution while the process of promoting etching is performed once. Accordingly, formation of hole 11 is performed uniformly in the direction of thickness of diaphragm 2. The symmetric property of the cross-sectional shape of hole 11 is improved by making the speed of revolution constant, thereby increasing the productivity. On the other hand, by rotating intermittently, the cross-sectional shape of hole 11 can be provided with a special shape, whereby a device which can cope with various types of culture solution 6 in which properties such as viscosity and surface tension are different is achieved.

Subsequently, as described in conjunction with FIG. 14 and FIG. 15, electrode 5 is formed on the entire wall surface of hole 11 and on lower surface 2B of diaphragm 2. At this time, by inclining substrate 1 at an angle with respect to the plane perpendicular to the ion-progressing direction, and then performing dry etching while rotating continuously, the shape of openings 11C, 11D of hole 11 become circular. The wall surface of hole 11 becomes inverted linear tapered shape. Therefore, electrode 5 can easily be formed without discontinuation.

By forming hole 11 according to the method as described above, electrode 5 can be formed easily without discontinuation, and the device for measuring extracellular potential having less possibility of discontinuation of electrode 5 is obtained.

Although diaphragm 2 is formed by performing dry etching on substrate 1 in the description above, this process is not necessary when diaphragm 2 has a sufficient strength. In other words, although substrate 1 has the first surface shared with diaphragm 2 and serves as a holding portion of diaphragm 2, it is also possible to hold the single piece of diaphragm 2 with other members instead of substrate 1.

As described above, the device for measuring extracellular potential and the method of manufacturing the same of the present invention is effective for a device which can measure the physico-chemical change occurred during activity of the cell efficiently and stably by a detecting electrode. It is used, for example, for medicine-screening in which the reaction generated by the cell using a chemical substance is detected and a pharmacologic effect on the cell is determined.

1. A device for measuring extracellular potential comprising:
a diaphragm provided with a depression formed of at least one curved surface for placing a cell on a first surface, and a through-hole having a first opening on the curved surface of the depression and a second opening being larger than the first opening on a second surface side opposing to the first surface; and
a detecting electrode provided at least on a wall surface of the through-hole.

2. The device for measuring extracellular potential according to claim 1,
wherein the ridge line of the wall surface of the through-hole extends linearly.

3. The device for measuring extracellular potential according to claim 1, further comprising a holding portion for holding the diaphragm.

4. The device for measuring extracellular potential according to claim 1, further comprising a substrate sharing the first surface with the diaphragm for holding the diaphragm.

5. The device for measuring extracellular potential according to claim 4,
wherein the substrate is formed of silicon.

6. The device for measuring extracellular potential according to claim 4,
wherein the substrate comprises two silicon layers, and a silicone dioxide layer interposed between the silicon layers, the silicon layers and the silicone dioxide layer being laminated together.

7. The device for measuring extracellular potential according to claim 1, wherein the detecting electrode includes at least one of gold and platinum.

8. A method of manufacturing a device for measuring extracellular potential comprising:
a step A of forming a resist mask provided with an etching hole on a first surface of a diaphragm;
a step B of forming a depression having at least one curved surface on the diaphragm for placing a cell by etching the diaphragm from over the resist mask;
a step D of forming a through-hole by dry etching due to ions from the side of the curved surface of the depression;
a step C of installing the diaphragm so as to incline by an angle larger than 0° with respect to a plane perpendicular to an ion-progressing direction, prior to the step D; and
a step E for forming a detecting electrode at least within the through-hole.

9. The method of manufacturing a device for measuring extracellular potential according to claim 8,
wherein after the step D, the diaphragm is rotated by a predetermined angle within the first surface, and the step D is further performed.

10. The method of manufacturing a device for measuring extracellular potential according to claim 9,
wherein the angle of rotation is 360°/n (n designates positive integer).

11. The method of manufacturing a device for measuring extracellular potential according to claim 8, wherein the diaphragm is installed so as to incline by an angle larger than 0° and at most 7° with respect to the plane perpendicular to the ion-progressing direction in the step C.

12. The method of manufacturing a device for measuring extracellular potential according to claim 8,
wherein a step F of promoting etching and a step G for forming a protective film on an inner wall etched in the step F are repeated alternately in the step D.

13. The method of manufacturing a device for measuring extracellular potential according to claim 12,
wherein gas containing any one of XeF₂, CF₄ and SF₆ is used for promoting etching in the step F, and gas containing any one of CHF₃ and C₄F₈ is used for forming the protective film in the step G.

14. The method of manufacturing a device for measuring extracellular potential according to claim 8, wherein the step D is performed while rotating the diaphragm within the first surface after the step C.

15. The method of manufacturing a device for measuring extracellular potential according to claim 14,
wherein a step F of promoting etching and a step G of forming a protective film on an inner wall etched in the step F are repeated alternatively in the step D, and the diaphragm is rotated at least by one revolution while the step F is performed once.

16. The method of manufacturing a device for measuring extracellular potential according to claim 14,
wherein the diaphragm is rotated at a constant speed.

17. The method of manufacturing a device for measuring extracellular potential according to claim 14,
wherein the diaphragm is rotated intermittently.
